(19) United States
(12) Patent Application Publication
(10) Pub. No.: US 2025/0314729 A1
(43) Pub. Date: Oct. 9, 2025

(54) Motion Estimation in Magnetic Resonance Imaging System

(71) Applicant: Siemens Healthineers AG, Forchheim (DE)

(72) Inventors: Yan Tu Huang, Shenzhen (CN); Hui Xin Tan, Shenzhen (CN); Nan Xiao, Shenzhen (CN); Ce Wang, Shenzhen (CN); Daniel Nicolas Splitthoff, Uttenreuth (DE); Daniel Polak, Erlangen (DE); Marcel Dominik Nickel, Herzogenaurach (DE); Tobias Kober, Heidelberg (DE); Tom Hilbert, Lausanne (CH)

(73) Assignee: Siemens Healthineers AG, Forchheim (DE)

(21) Appl. No.: 19/174,073

(22) Filed: Apr. 9, 2025

(30) Foreign Application Priority Data
Apr. 9, 2024 (CN) 202410437107.6

(51) Int. Cl. G01R 33/565; G01R 33/56; G01R 33/561

(52) U.S. Cl. CPC G01R 33/56509; G01R 33/5608; G01R 33/5611; G01R 33/5615

(57) ABSTRACT

A motion estimation method and apparatus for magnetic resonance imaging and a magnetic resonance imaging system. The method includes: calculating a rigid body motion vector of a subject for each of a most recent N imaging excitations; according to pilot tone (PT) data of each channel acquired for the most recent N imaging excitations and N rigid body motion vectors calculated for the most recent N imaging excitations, calculating a linear transformation model for transforming from the PT data of each channel acquired for the most recent N imaging excitations to the N rigid body motion vectors; and for each subsequent imaging excitation, according to PT data of each channel acquired during an echo duration of each echo and the linear transformation model, calculating a rigid body motion vector of the subject for each echo of each imaging excitation.

### TECHNICAL FIELD

The present disclosure relates to the technical field of magnetic resonance (MR), in particular to a motion estimation method and apparatus for magnetic resonance imaging and a magnetic resonance imaging system.

### BACKGROUND

In the MR imaging process, in order to obtain clear MR images for clinical diagnosis, a subject is required to remain motionless during the scanning process. However, the motion of human body parts or organs cannot be completely avoided, which may cause motion artifacts in MR images.

In order to reduce the motion artifacts in MR images, a linear scout accelerated retrospective motion estimation and reduction (SAMER) scheme has recently been proposed. In this scheme, before each imaging scan, a scout scan is first performed to obtain scout data, and multiple imaging excitations are then performed to obtain imaging data and guidance data; after each imaging excitation, a rigid body motion vector corresponding to each imaging excitation is obtained by performing linear calculations on the scout data and the guidance data, wherein for any imaging excitation, such as the nth (n is an integer greater than 1) imaging excitation, the similarity between the guidance data of the nth imaging excitation and the guidance data of each of the previous imaging excitations is calculated, and the rigid body motion vector corresponding to the imaging excitation with the highest similarity is used to initialize the rigid body motion vector corresponding to the nth imaging excitation, wherein the rigid body motion vector corresponding to the 1st imaging excitation is initialized to 0. The disadvantages of this scheme are as follows: Firstly, the temporal resolution is low, and the rigid body motion vector is only calculated once for one imaging excitation. For example, currently, the rigid body motion vector is calculated once every 2 seconds or so. Secondly, the calculated rigid body motion vectors are not sufficiently accurate, especially when the subject moves very little or does not move, because the acquired scout images will have noise, and the acquired guidance data will also have noise. When the subject moves very little or does not move, the noise of the scout images and guidance data will have a more significant impact on the accuracy of the rigid body motion vectors.

### SUMMARY

In view of this, an aspect of the present disclosure proposes a motion estimation method and apparatus for MR imaging to improve the temporal resolution, accuracy and robustness of motion estimation in MR imaging; another aspect proposes an MR imaging system to improve the temporal resolution, accuracy and robustness of motion estimation in MR imaging; and still another aspect proposes a computer program product, a computer-readable storage medium and an electronic device to improve the temporal resolution, accuracy and robustness of motion estimation in MR imaging.

A motion estimation method for magnetic resonance imaging, the method comprising:
calculating a rigid body motion vector of a subject for each of the most recent N imaging excitations, where N is a preset integer greater than 1;
calculating, according to pilot tone (PT) data of each channel acquired for the most recent N imaging excitations and N rigid body motion vectors calculated for the most recent N imaging excitations, a linear transformation model for transforming from the PT data of each channel acquired for the most recent N imaging excitations to the N rigid body motion vectors; wherein a PT scan is continuously performed on the subject during an imaging scan of the subject;
for each subsequent imaging excitation, according to PT data of each channel acquired during an echo duration of each echo and the linear transformation model, calculating a rigid body motion vector of the subject for each echo of each imaging excitation.

Calculating the rigid body motion vector of the subject for each of the most recent N imaging excitations comprises:
acquiring scout data, wherein a scout scan is performed on the subject before the start of the first imaging scan of the subject;
for each of the most recent N imaging excitations, acquiring pilot tone (PT) data of each channel while acquiring imaging data, and acquiring guidance data during an echo duration of a plurality of preset echoes of each imaging excitation;
calculating the rigid body motion vector of the subject for each of the most recent N imaging excitations according to the scout data and the guidance data acquired for each of the most recent N imaging excitations.

Calculating the rigid body motion vector of the subject for each of the most recent N imaging excitations according to the scout data and the guidance data acquired for each of the most recent N imaging excitations comprises:
by using a linear scout accelerated retrospective motion estimation and reduction (SAMER) method, according to the scout data and the guidance data acquired for each of the most recent N imaging excitations, calculating the rigid body motion vector of the subject for each of the most recent N imaging excitations.

Calculating, according to the PT data of each channel acquired for the most recent N imaging excitations and N rigid body motion vectors calculated for the most recent N imaging excitations, the linear transformation model for transforming from the PT data of each channel acquired for the most recent N imaging excitations to the N rigid body motion vectors comprises:
for each of the most recent N imaging excitations, selecting, from the PT data of each channel for the imaging excitation, PT data of each channel during a period from the start of acquiring the first piece of guidance data to the end of acquiring the last piece of guidance data, and averaging the PT data of each selected channel to obtain a PT mean value of each channel for the imaging excitation;
according to the PT mean value of each channel for each of the most recent N imaging excitations and the N rigid body motion vectors, calculating the linear transformation model for transforming from the PT data of each channel acquired for the most recent N imaging excitations to the N rigid body motion vectors.

Calculating, for each subsequent imaging excitation, the rigid body motion vector of the subject for each echo of each imaging excitation according to the PT data of each channel acquired during the echo duration of each echo and the linear transformation model comprises:
for each echo, averaging the PT data of each channel corresponding to the echo to obtain the PT mean value of each channel for the echo, and calculating the rigid body motion vector of the subject for the echo according to the PT mean value of each channel for the echo and the linear transformation model.

After calculating the rigid body motion vector of the subject for each echo of each imaging excitation, the method further comprises:
when all imaging excitations for the imaging scan of the subject have been completed,
for any imaging excitation, when the rigid body motion vector of the subject for the imaging excitation is calculated, performing motion correction on imaging data of the imaging excitation by using the rigid body motion vector for the imaging excitation;
for any imaging excitation, when the rigid body motion vector of the subject for each echo of the imaging excitation is calculated, performing motion correction on imaging data of each echo acquired for the imaging excitation by using the rigid body motion vector for the echo.

After calculating the rigid body motion vector of the subject for each of the most recent N imaging excitations, the method further comprises:
for each of the most recent N imaging excitations, according to the rigid body motion vector of the subject for the imaging excitation, or according to the rigid body motion vectors of the subject for the most recent M imaging excitations, predicting a position to which the subject will move in a next imaging excitation; and adjusting scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N;
and/or, after calculating the rigid body motion vector of the subject for each echo of each imaging excitation, the method further comprises:
for each imaging excitation, according to the rigid body motion vector of the subject for each echo of the imaging excitation, or according to the rigid body motion vector of the subject for each echo of the most recent Q imaging excitations, predicting a position to which the subject will move in a next imaging excitation; and adjusting scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

A motion estimation apparatus for magnetic resonance imaging, the apparatus comprising:
a first rigid body motion vector calculation module, configured to calculate a rigid body motion vector of a subject for each of the most recent N imaging excitations, where N is a preset integer greater than 1;
a linear transformation model calculation module, configured to calculate, according to PT data of each channel acquired for the most recent N imaging excitations and N rigid body motion vectors calculated for the most recent N imaging excitations, a linear transformation model for transforming from the PT data of each channel acquired for the most recent N imaging excitations to the N rigid body motion vectors; wherein a PT scan is continuously performed on the subject during an imaging scan of the subject;
a second rigid body motion vector calculation module, configured to calculate, for each subsequent imaging excitation, a rigid body motion vector of the subject for each echo of each imaging excitation according to PT data of each channel acquired during an echo duration of each echo and the linear transformation model.

The first rigid body motion vector calculation module calculating the rigid body motion vector of the subject for each of the most recent N imaging excitations comprises:
acquiring scout data, wherein a scout scan is performed on the subject before the start of the first imaging scan of the subject;
for each of the most recent N imaging excitations, acquiring pilot tone (PT) data of each channel while acquiring imaging data, and also acquiring guidance data during an echo duration of a plurality of preset echoes of each imaging excitation;
calculating the rigid body motion vector of the subject for each of the most recent N imaging excitations according to the scout data and the guidance data acquired for each of the most recent N imaging excitations.

The linear transformation model calculation module calculating, according to the PT data of each channel acquired for the most recent N imaging excitations and the N rigid body motion vectors calculated for the most recent N imaging excitations, the linear transformation model for transforming from the PT data of each channel acquired for the most recent N imaging excitations to the N rigid body motion vectors comprises:
for each of the most recent N imaging excitations, selecting, from the PT data of each channel for the imaging excitation, PT data of each channel during a period from the start of acquiring the first piece of guidance data to the end of acquiring the last piece of guidance data, and averaging the PT data of each selected channel to obtain a PT mean value of each channel for the imaging excitation;
according to the PT mean value of each channel for each of the most recent N imaging excitations and the N rigid body motion vectors, calculating the linear transformation model for transforming from the PT mean value of each channel for the imaging excitation to the N rigid body motion vectors.

The second rigid body motion vector calculation module calculating, for each subsequent imaging excitation, the rigid body motion vector of the subject for each echo of each imaging excitation according to the PT data of each channel acquired during the echo duration of each echo and the linear transformation model comprises:
for each echo, averaging the PT data of each channel corresponding to the echo to obtain the PT mean value of each channel for the echo, and calculating the rigid body motion vector of the subject for the echo according to the PT mean value of each channel for the echo and the linear transformation model.

The apparatus further comprises: a motion correction module, configured to:
when all imaging excitations for the imaging scan of the subject have been completed,
for any imaging excitation, when the rigid body motion vector of the subject for the imaging excitation is calculated, perform motion correction on imaging data of the imaging excitation by using the rigid body motion vector for the imaging excitation;
for any imaging excitation, when the rigid body motion vector of the subject for each echo of the imaging excitation is calculated, perform motion correction on imaging data of each echo acquired for the imaging excitation by using the rigid body motion vector for the echo.

The apparatus further comprises: a scanning parameter adjustment module, configured to:
after the first rigid body motion vector calculation module calculates the rigid body motion vector of the subject for each of the most recent N imaging excitations, for each of the most recent N imaging excitations, according to the rigid body motion vector of the subject for the imaging excitation, or according to the rigid body motion vectors of the subject for the most recent M imaging excitations, predict a position to which the subject will move in a next imaging excitation; and adjust scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N;
and/or, configured to: after the second rigid body motion vector calculation module calculates the rigid body motion vector of the subject for each echo of each imaging excitation, for each imaging excitation, according to the rigid body motion vector of the subject for each echo of the imaging excitation, or according to the rigid body motion vector of the subject for each echo of the most recent Q imaging excitations, predict a position to which the subject will move in a next imaging excitation; and adjust scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

A magnetic resonance imaging system, the magnetic resonance imaging system comprising any one of the motion estimation apparatuses for magnetic resonance imaging as described above.

In the aspects of the present disclosure, during an MR imaging scan of a subject, a rigid body motion vector of the subject is first calculated once (1 time) for each imaging excitation, and then, according to PT data acquired for the most recent N imaging excitations and the calculated N rigid body motion vectors, a linear transformation model therebetween is calculated. After that, for each subsequent imaging excitation, 1 rigid body motion vector is calculated for each echo according to the PT data acquired for each echo and the linear transformation model, thereby greatly improving the time resolution, accuracy and robustness of motion estimation of the subject during MR imaging, and ultimately greatly improving the accuracy of the reconstructed MR image and increasing the speed of image reconstruction.

### BRIEF DESCRIPTION OF THE DRAWINGS

Preferred aspects of the present disclosure will be described in detail below with reference to the drawings to give an ordinary person skilled in the art a clearer understanding of the above-mentioned and other features and advantages of the present disclosure. In the drawings:

FIG. 1 is a flowchart of a motion estimation method for MR imaging provided in an aspect of the present disclosure;

FIG. 2 is a schematic diagram showing a comparison of PT data before and after radio frequency interference suppression processing in an application example of the present disclosure;

FIG. 3 is a schematic diagram showing excitation and acquisition during an imaging scanning process in an application example of the present disclosure;

FIG. 4 is a schematic diagram showing a comparison of reconstructed images obtained by using the prior art and an aspect of the present disclosure, respectively, when a volunteer remained substantially motionless during a brain imaging scan;

FIG. 5 is a schematic diagram showing a comparison of rigid body motion vectors obtained by solution three and solution two in FIG. 4;

FIG. 6 is a schematic diagram showing the difference between rigid body motion vectors obtained by solution three and solution two for a time of 0-122 s in FIG. 5;

FIG. 7 is a schematic diagram showing a comparison of reconstructed images obtained by using the prior art and the aspect of the present disclosure, respectively, when the volunteer moved in a step-by-step manner during a brain imaging scan;

FIG. 8 is a schematic diagram showing a comparison of rigid body motion vectors obtained by solution three and solution two in FIG. 7;

FIG. 9 is a schematic diagram showing an enlargement of rigid body motion vectors obtained by solution three and solution two for a time of 96-106 s in FIG. 8;

FIG. 10 is a schematic diagram showing a comparison of reconstructed images obtained by using the prior art and the aspect of the present disclosure, respectively, when the volunteer kept talking (that is, the mouth kept moving) during a brain imaging scan;

FIG. 11 is a schematic diagram showing a comparison of rigid body motion vectors obtained by solution three and solution two in FIG. 10;

FIG. 12 is a schematic diagram showing an amplification of rigid body motion vectors obtained by solution three and solution two for a time of 80-90 s in FIG. 11; and

FIG. 13 is a schematic structural diagram of a motion estimation apparatus for MR imaging provided in an aspect of the present disclosure.

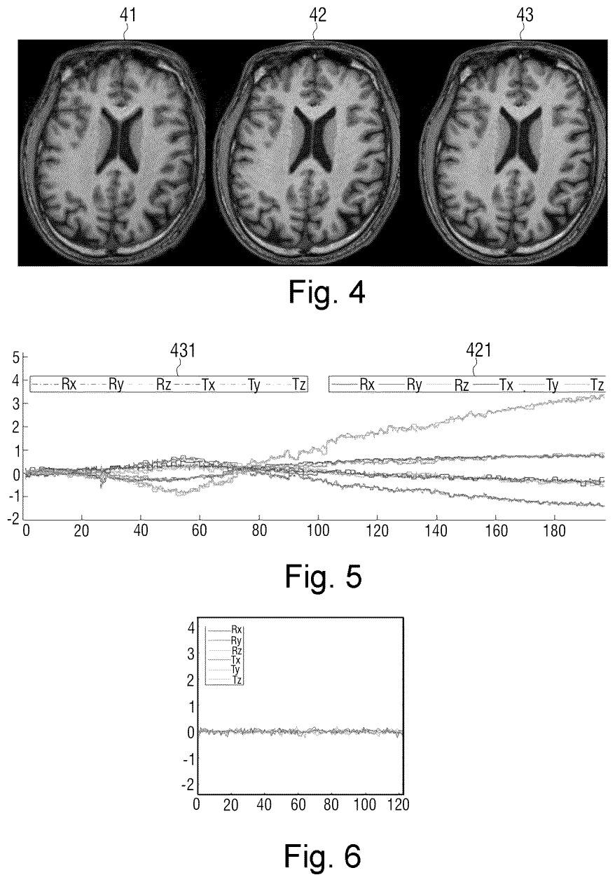

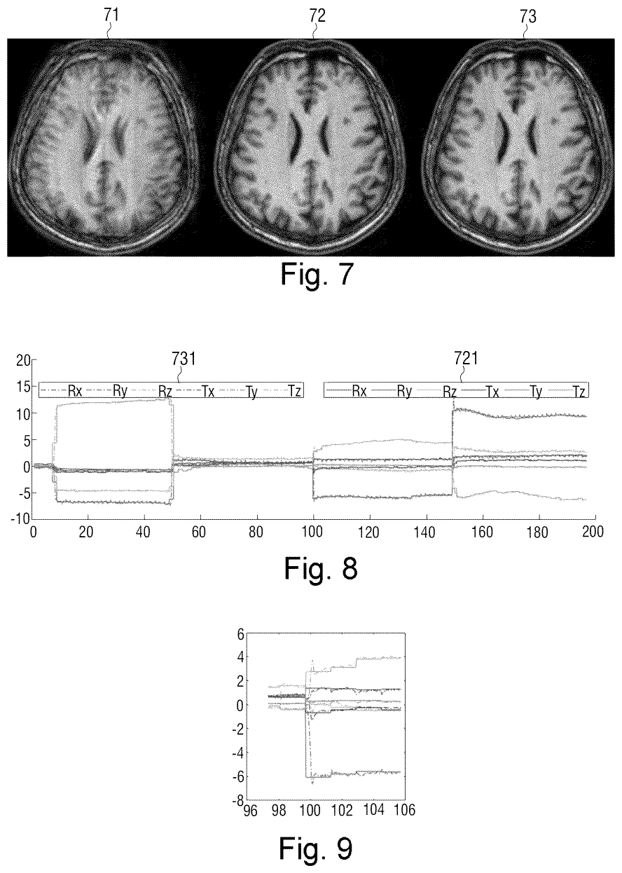

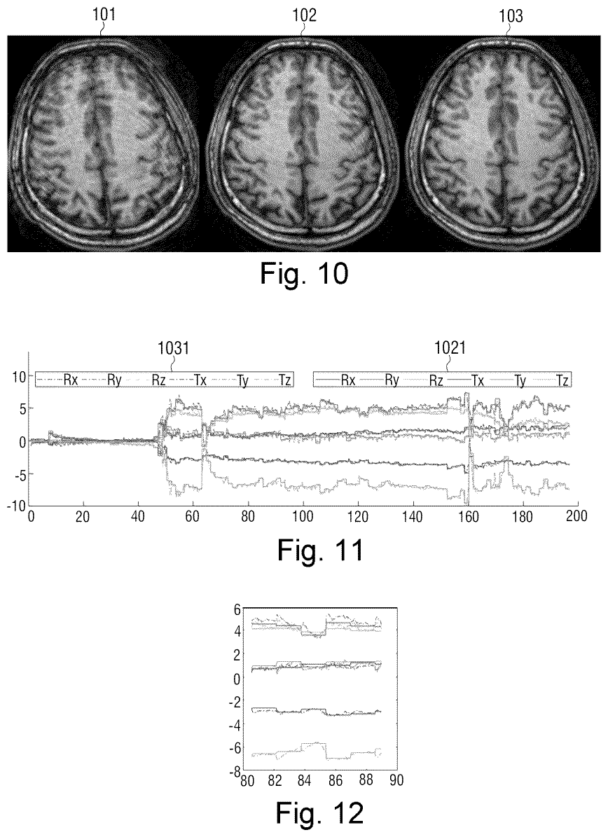

In the figures, the reference numerals are as follows:

| Label | Meaning |
|---|---|
| 101- | Steps |
| 103 | |
| 21 | Collected PT data of each channel |
| 22 | Corresponding PT data obtained after the PT data of |
| | each channel in 21 is subjected to radio frequency |
| | interference suppression processing |
| 31 | Scout scan |
| 32 | First imaging excitation |
| 321 | PT data acquired in the first imaging excitation |
| 33 | Part of acquired PT data |
| 331 | Enlarged image of 33 |
| 301- | PT data acquired in one echo |
| 304 | |
| 41 | Reconstructed image obtained by performing image |
| | reconstruction using an existing SENSE method without |
| | motion estimation and correction when a volunteer |
| | remained substantially motionless during a brain imaging scan |
| 42 | Reconstructed image obtained by performing motion estimation, |
| | correction, and image reconstruction using an existing linear |
| | SAMER method when the volunteer remained substantially |
| | motionless during a brain imaging scan |
| 43 | Reconstructed image obtained by performing motion estimation, |
| | correction, and image reconstruction using a solution provided in |
| | an aspect of the present disclosure when the volunteer remained |
| | substantially motionless during a brain imaging scan |
| 431 | Dashed lines of different colors respectively corresponding to |
| | rotation parameter values of three directions and translation |
| | parameter values of the three directions of rigid body motion |
| | vectors obtained by solution three in FIG. 4 |
| 421 | Solid lines of different colors respectively corresponding to |
| | rotation parameter values of the three directions and translation |
| | parameter values of the three directions of rigid body motion |
| | vectors obtained by solution two in FIG. 4 |
| Rx | Rotation parameter value in an x direction |
| Ry | Rotation parameter value in a y direction |
| Rz | Rotation parameter value in a z direction |
| Tx | Translation parameter value in the x direction |
| Ty | Translation parameter value in the y direction |
| Tz | Translation parameter value in the z direction |
| 71 | Reconstructed image obtained by performing image |
| | reconstruction using the existing SENSE method without motion |
| | estimation and correction when the volunteer moved |
| | in a step-by-step manner during a brain imaging scan |
| 72 | Reconstructed image obtained by performing motion estimation, |
| | correction, and image reconstruction using the existing linear |
| | SAMER method when the volunteer moved in a step-by-step |
| | manner during a brain imaging scan |
| 73 | Reconstructed image obtained by performing motion estimation, |
| | correction, and image reconstruction using the solution provided |
| | in the aspect of the present disclosure when the volunteer moved |
| | in a step-by-step manner during a brain imaging scan |
| 731 | Dashed lines of different colors respectively corresponding to |
| | rotation parameter values of three directions and translation |
| | parameter values of the three directions of rigid body motion |
| | vectors obtained by solution three in FIG. 7 |
| 721 | Solid lines of different colors respectively corresponding to |
| | rotation parameter values of the three directions and translation |
| | parameter values of the three directions of rigid body motion |
| | vectors obtained by solution two in FIG. 7 |
| 101 | Reconstructed image obtained by performing image |
| | reconstruction using the existing SENSE method without motion |
| | estimation and correction when the volunteer kept talking |
| | (that is, the mouth kept moving) during a brain imaging scan |
| 102 | Reconstructed image obtained by performing motion estimation, |
| | correction, and image reconstruction using the existing linear |
| | SAMER method when the volunteer kept talking (that is, the |
| | mouth kept moving) during a brain imaging scan |
| 103 | Reconstructed image obtained by performing motion estimation, |
| | correction, and image reconstruction using the solution provided |
| | in the aspect of the present disclosure when the volunteer kept |
| | talking (that is, the mouth kept moving) during a brain imaging |
| | scan |
| 1031 | Dashed lines of different colors respectively corresponding to |
| | rotation parameter values of three directions and translation |
| | parameter values of the three directions of rigid body motion |
| | vectors obtained by solution three in FIG. 10 |
| 1021 | Solid lines of different colors respectively corresponding to |
| | rotation parameter values of the three directions and translation |
| | parameter values of the three directions of rigid body motion |
| | vectors obtained by solution two in FIG. 10 |
| 130 | Motion estimation apparatus for MR imaging |
| 131 | First rigid body motion vector calculation module |
| 132 | Linear transformation model calculation module |
| 133 | Second rigid body motion vector calculation module |
| 134 | Motion correction module |
| 135 | Scanning parameter adjustment module |

### DETAILED DESCRIPTION

To clarify the objectives, technical solutions, and advantages of the present disclosure, the present disclosure will be explained in further detail below through aspects.

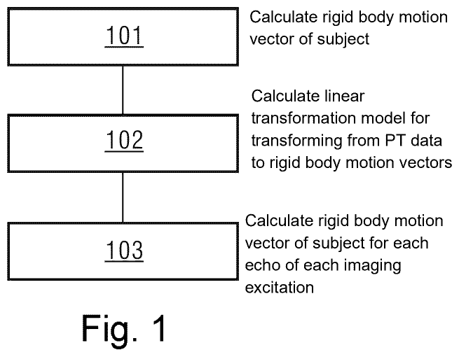

FIG. 1 is a flowchart of a motion estimation method for MR imaging provided in an aspect of the present disclosure, and specific steps thereof are as follows:

Step 101: Calculate a rigid body motion vector of a subject for each of the most recent N imaging excitations, where N is a preset integer greater than 1.

There are mature algorithms for the specific implementation of step 101, such as a linear SAMER algorithm or a motion parameter estimating dense net (MoPED) based algorithm, or a coil-mixing based algorithm, or a navigator based algorithm, or a camera system based algorithm (e.g., acquiring the image of the subject through a camera, and calculating the rigid body motion vector of the subject using an image processing algorithm), or the like. The aspect of the present disclosure does not limit the specific algorithm used in step 101.

In an optional aspect, step 101 specifically includes: acquiring scout data, wherein a scout scan is performed on the subject before the start of the first imaging scan of the subject; for each of the most recent N imaging excitations, acquiring pilot tone (PT) data of each channel while acquiring imaging data, and acquiring guidance data during an echo duration of a plurality of preset echoes of each imaging excitation; calculating the rigid body motion vector of the subject for each of the most recent N imaging excitations according to the scout data and the guidance data acquired for each of the most recent N imaging excitations.

A PT scan is continuously performed on the subject during an imaging scan of the subject. Guidance data refers to data that can clearly characterize the motion state of the subject. Usually, according to a target scanning area of an imaging scan, it is determined in advance based on experience, etc., which echo data of the echoes at which positions in the echoes generated by each imaging excitation can clearly characterize the motion state of the subject, and the echo data of the echoes at these positions (these positions are usually not continuous) are used as the guidance data.

For example, when the linear SAMER algorithm is used, in step 101, calculating the rigid body motion vector of the subject for each of the most recent N imaging excitations according to the scout data and the guidance data acquired for each of the most recent N imaging excitations includes: by using the linear SAMER method, according to the scout data and the guidance data acquired for each of the most recent N imaging excitations, calculating the rigid body motion vector of the subject for each of the most recent N imaging excitations.

In order to more accurately track the motion of the subject, when a PT transmitter is placed, the PT transmitter should be placed at a position that best reflects the real-time motion state of the subject. For example, when performing an imaging scan on the subject's brain, in order to more accurately track the subject's head motion, the PT transmitter may be placed at the back of the subject's head.

In an optional aspect, in step 101, acquiring the PT data of each channel includes: acquiring PT data of each channel within a preset range of each echo center. The preset range may be set based on experience or the like in advance.

In an optional aspect, after acquiring the PT data of each channel, step 101 further includes: performing radio frequency interference suppression processing on the PT data of each channel. Radio frequency interference suppression is a mature technology, and the aspect of the present disclosure does not limit the radio frequency interference suppression algorithm used.

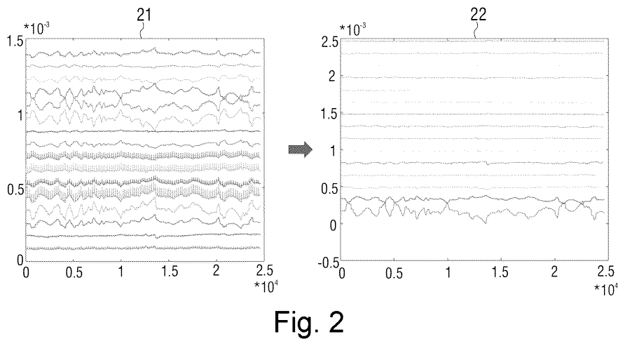

FIG. 2 is a schematic diagram showing a comparison of PT data before and after radio frequency interference suppression processing in an application example of the present disclosure. In the figure, 21 is the PT data of each channel obtained by acquisition, i.e., original PT data without RF interference processing, wherein each curve corresponds to the PT data acquired during the echo duration of an echo of an imaging excitation of an imaging scan; 22 is the corresponding PT data obtained after the PT data of each channel in 21 is subjected to RF interference suppression processing. By comparing various curves in 22 with those in 21, it can be seen that after the RF interference suppression processing, the RF interference in the PT data is greatly eliminated. The abscissa in 21 and 22 represents the sampling point number of the PT data, and the ordinate is the relative signal intensity of the PT data.

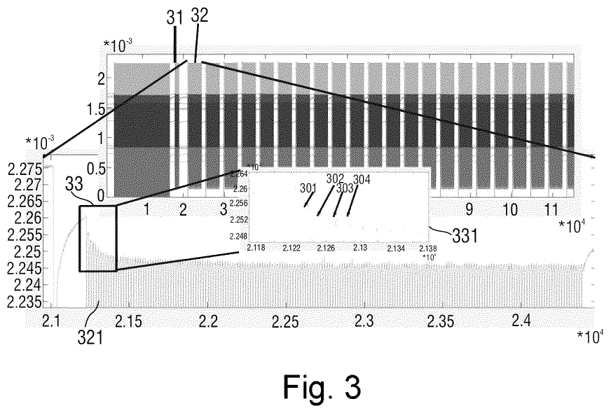

FIG. 3 is a schematic diagram showing excitation and acquisition during an imaging scanning process in an application example of the present disclosure, wherein 31 corresponds to a scout scan, 32 corresponds to the first imaging excitation, 321 is PT data acquired in the first imaging excitation, 331 is a schematic diagram showing an enlargement of part 33 of 321, and 301-304 each correspond to PT data acquired in one echo. The abscissa in FIG. 3 represents the sampling point number, and the ordinate represents the relative signal intensity.

Step 102: According to PT data of each channel acquired for the most recent N imaging excitations and N rigid body motion vectors calculated for the most recent N imaging excitations, calculate a linear transformation model for transforming from the PT data of each channel acquired for the most recent N imaging excitations to the N rigid body motion vectors; wherein a PT scan is continuously performed on the subject during an imaging scan of the subject.

In an optional aspect, step 102 specifically includes:

Step 1021: For each of the most recent N imaging excitations, select, from the PT data of each channel for the imaging excitation, PT data of each channel during a period from the start of acquiring the first piece of guidance data to the end of acquiring the last piece of guidance data, and average the PT data of each selected channel separately to obtain a PT mean value of each channel for the imaging excitation.

For example, there are p channels in total. For any one (set as the nth imaging excitation) of the most recent N imaging excitations, in total, PT data of p channels is acquired. For the PT data of each channel, PT data during a period from the start of acquiring the first guidance data to the end of acquiring the last guidance data is selected therefrom (because the PT data during this period can better characterize the motion state of the subject). Then, for each channel, the PT data selected from the channel are averaged to obtain a PT mean value of the channel; that is, each channel corresponds to one PT mean value, and a total of p PT mean values are obtained.

Step 1022: According to the PT mean value of each channel for each of the most recent N imaging excitations and the N rigid body motion vectors, calculate the linear transformation model for transforming from the PT mean value of each channel for the imaging excitation to the N rigid body motion vectors.

The rigid body motion vector is usually a 6*1 vector, that is, it includes 6 parameters, three translation parameters and three rotation parameters. The three translation parameters are translation parameters in mutually perpendicular x, y, and z directions, and the three rotation parameters are rotation parameters in the mutually perpendicular x, y, and z directions.

In an optional aspect, the linear transformation model is a linear transformation matrix.

Assuming that there are p channels in total, the PT mean values of the respective channels for each of the most recent N imaging excitations form a p*N matrix P_{g}, and the N rigid body motion vectors corresponding to the most recent N imaging excitations form a 6*N matrix Aₛ. According to QP_{g}=Aₛ, it can be known that: Q=AₛP_{g}, where Q is a linear transformation matrix with a size of 6*p.

Step 103: Calculate, for each subsequent imaging excitation, a rigid body motion vector of the subject for each echo of each imaging excitation according to PT data of each channel acquired during an echo duration of each echo and the linear transformation model.

In an optional aspect, after calculating the rigid body motion vector of the subject for each echo of each imaging excitation, step 103 further includes: performing radio frequency interference suppression processing on the PT data of each channel corresponding to the echo of the imaging excitation.

In an optional aspect, step 103 specifically includes: for each echo, averaging the PT data of each channel corresponding to the echo to obtain the PT mean value of each channel for the echo, and calculating the rigid body motion vector of the subject for the echo according to the PT mean value of each channel for the echo and the linear transformation model obtained in step 102.

That is, when the linear transformation model in step 102 is a linear transformation matrix Q, and the PT mean value of each channel for a certain echo is P_{g}, then the rigid body motion vector As of the subject for the echo is: As=QP_{g}.

It can be seen that in step 103, 1 rigid body motion vector is calculated for each echo of each imaging excitation of the imaging scan.

In the above aspect, during the MR imaging scan of the subject, the rigid body motion vector of the subject is first calculated once (1 time) for each imaging excitation, and then, according to the PT data acquired for the most recent N imaging excitations and the calculated N rigid body motion vectors, the linear transformation model therebetween is calculated. After that, for each subsequent imaging excitation, 1 rigid body motion vector is calculated for each echo according to the PT data acquired for each echo and the linear transformation model, thereby greatly improving the time resolution, accuracy, and robustness of motion estimation of the subject during MR imaging. Even if the subject's motion is very small, it can be sensitively tracked with higher precision and robustness, which ultimately greatly improves the accuracy of the reconstructed MR image and increasing the speed of image reconstruction, causing the image reconstruction time to meet clinical requirements.

In an optional aspect, after step 103, the method further includes: when all imaging excitations for the imaging scan of the subject have been completed, for any imaging excitation of the imaging scan, when the rigid body motion vector of the subject for the imaging excitation is calculated (that is, when only 1 rigid body motion vector is calculated for the imaging excitation), performing motion correction on imaging data of the imaging excitation by using the rigid body motion vector for the imaging excitation (for example: performing translation correction on each piece of imaging data for the imaging excitation in the three directions by using the translation parameter values in the three directions of the rigid body motion vector obtained for the imaging excitation, and then performing rotation correction on each piece of imaging data for the imaging excitation in the three directions by using the rotation parameter values in the three directions of the rigid body motion vector obtained for the imaging excitation); for any imaging excitation of the imaging scan, when the rigid body motion vector of the subject for each echo of the imaging excitation is calculated (that is, when only 1 rigid body motion vector is calculated for each echo of the imaging excitation), performing motion correction on imaging data of each echo acquired for the imaging excitation by using the rigid body motion vector for the echo (for example: for each echo of the imaging excitation, performing translation correction on each piece of imaging data for the echo in the three directions by using the translation parameter values in the three directions of the rigid body motion vector for the echo, and then performing rotation correction on each piece of imaging data for the echo in the three directions by using the rotation parameter values in the three directions of the rigid body motion vector for the echo).

After motion correction has been performed on the imaging data for all imaging excitations of the imaging scan, image reconstruction is performed according to the motion-corrected imaging data to obtain a final MR image. Image reconstruction is a mature technology, and the aspect of the present disclosure does not limit the image reconstruction algorithm used. For example, a non-uniform fast Fourier transform (NUFFT) or the like may be used.

In addition, it is also possible to use SENSitivity Encoding (SENSE)+motion model in the linear SAMER to perform motion correction and image reconstruction on the imaging data. The use of the NUFFT method can greatly improve the speed of image reconstruction compared with SENSE+motion model.

In an optional aspect, in order to further improve the accuracy of motion estimation, after calculating the rigid body motion vector of the subject for each of the most recent N imaging excitations, step 101 further includes: for each of the most recent N imaging excitations, according to the rigid body motion vector of the subject for the imaging excitation, or according to the rigid body motion vectors of the subject for the most recent M imaging excitations, predicting a position to which the subject will move in a next imaging excitation; and adjusting scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N;
and/or, after calculating the rigid body motion vector of the subject for each echo of each imaging excitation, step 103 further includes: for each imaging excitation, according to the rigid body motion vector of the subject for each echo of the imaging excitation, or according to the rigid body motion vector of the subject for each echo of the most recent Q imaging excitations, predicting a position to which the subject will move in a next imaging excitation; and adjusting scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

Several application examples of the present disclosure will be compared with the prior art below:

FIG. 4 shows reconstructed images respectively obtained by using the following three solutions of the prior art and an aspect of the present disclosure when a volunteer remained substantially motionless during a brain imaging scan:

Solution one: Without performing motion estimation and correction, the existing SENSE method is used to perform image reconstruction to obtain a reconstructed image 41.

Solution two: The existing linear SAMER method is used to perform motion estimation, correction, and image reconstruction to obtain a reconstructed image 42.

Solution three: The solution provided in the aspect of the present disclosure is used to perform motion estimation, correction, and image reconstruction to obtain a reconstructed image 43.

It can be seen from 41-43 that the reconstructed image corresponding to 43 has clearer details.

FIG. 5 is a schematic diagram showing a comparison between rigid body motion vectors obtained by solution three and solution two in FIG. 4, where the abscissa is time, in units of s (seconds), and the ordinate is a parameter value of the rigid body motion vector, in units of mm (millimeter) when it is a translation parameter, and mm/deg (millimeter/degree) when it is a rotation parameter. In the figure:

6 dashed lines of different colors in 431 correspond to the rotation parameter values of the three directions and the translation parameter values of the three directions of rigid body motion vectors obtained by solution three, respectively. Specifically:
the dark blue dashed line corresponds to the rotation parameter value Rx in the x direction;
the red dashed line corresponds to the rotation parameter value Ry in the y direction;
the yellow dashed line corresponds to the rotation parameter value Rz in the z direction;
the purple dashed line corresponds to the translation parameter value Tx in the x direction;
the green dashed line corresponds to the translation parameter value Ty in the y direction;
the light blue dashed line corresponds to the translation parameter value Tz in the z direction.

6 solid lines of different colors in 421 correspond to the rotation parameter values of the three directions and the translation parameter values of the three directions of rigid body motion vectors obtained by solution two, respectively. Specifically:
the dark blue solid line corresponds to the rotation parameter value Rx in the x direction;
the red solid line corresponds to the rotation parameter value Ry in the y direction;
the yellow solid line corresponds to the rotation parameter value Rz in the z direction;
the purple solid line corresponds to the translation parameter value Tx in the x direction;
the green solid line corresponds to the translation parameter value Ty in the y direction;
the light blue solid line corresponds to the translation parameter value Tz in the z direction.

In order to more clearly compare the rigid body motion vectors obtained by solution three and solution two in FIG. 5, FIG. 6 is a schematic diagram showing the difference between rigid body motion vectors obtained by solution three and solution two for a time of 0-122 s in FIG. 5, where the abscissa is time, in units of s, and the ordinate is the difference between the rigid body motion vectors obtained by solution three and solution two at each time, in units of mm (corresponding to a translation parameter) or mm/deg (corresponding to a rotation parameter). The dark blue solid line corresponds to the difference Rx of the rotation parameter value in the x direction, the red solid line corresponds to the difference Ry of the rotation parameter value in the y direction, the yellow solid line corresponds to the difference Rz of the rotation parameter value in the z direction, the purple solid line corresponds to the difference Tx of the translation parameter value in the x direction, the green solid line corresponds to the difference Ty of the translation parameter value in the y direction, and the light blue solid line corresponds to the difference Tz of the translation parameter value in the z direction.

It can be seen from FIG. 6 that the difference in the rigid body motion vector obtained by solution three and solution two substantially corresponds to the noise filtered out by solution three relative to solution two (such as noise in the scout data or guidance data), reflecting the accuracy of the rigid body motion vector obtained by the aspect of the present disclosure.

FIG. 7 shows reconstructed images respectively obtained by using the following three solutions of the prior art and the aspect of the present disclosure when the volunteer moved in a step-by-step manner during a brain imaging scan:

Solution one: Without performing motion estimation and correction, the existing SENSE method is used to perform image reconstruction to obtain a reconstructed image 71.

Solution two: The existing linear SAMER method is used to perform motion estimation, correction, and image reconstruction to obtain a reconstructed image 72.

Solution three: The solution provided in the aspect of the present disclosure is used to perform motion estimation, correction, and image reconstruction to obtain a reconstructed image 73.

It can be seen from 71-73 that the reconstructed image corresponding to 73 has clearer details.

FIG. 8 is a schematic diagram showing a comparison between rigid body motion vectors obtained by solution three and solution two in FIG. 7, where the abscissa is time, in units of s, and the ordinate is a parameter value of the rigid body motion vector, in units of mm (corresponding to a translation parameter) or mm/deg (corresponding to a rotation parameter). In the figure:

6 dashed lines of different colors in 731 correspond to the rotation parameter values of the three directions and the translation parameter values of the three directions of rigid body motion vectors obtained by solution three, respectively. Specifically:
the dark blue dashed line corresponds to the rotation parameter value Rx in the x direction;
the red dashed line corresponds to the rotation parameter value Ry in the y direction;
the yellow dashed line corresponds to the rotation parameter value Rz in the z direction;
the purple dashed line corresponds to the translation parameter value Tx in the x direction;
the green dashed line corresponds to the translation parameter value Ty in the y direction;
the light blue dashed line corresponds to the translation parameter value Tz in the z direction.

6 solid lines of different colors in 721 correspond to the rotation parameter values of the three directions and the translation parameter values of the three directions of rigid body motion vectors obtained by solution two, respectively. Specifically:
the dark blue solid line corresponds to the rotation parameter value Rx in the x direction;
the red solid line corresponds to the rotation parameter value Ry in the y direction;
the yellow solid line corresponds to the rotation parameter value Rz in the z direction;
the purple solid line corresponds to the translation parameter value Tx in the x direction;
the green solid line corresponds to the translation parameter value Ty in the y direction;
the light blue solid line corresponds to the translation parameter value Tz in the z direction.

In order to more clearly compare the rigid body motion vectors obtained by solution three and solution two in FIG. 8, FIG. 9 is a schematic diagram showing an enlargement of the rigid body motion vectors obtained by solution three and solution two for a time of 96-106 s in FIG. 8. It can be seen from FIG. 9 that the rigid body motion vector obtained by solution three changes more smoothly and has a higher temporal resolution of the motion state, which is more in line with the characteristics of the volunteer's stepping motion.

FIG. 10 shows reconstructed images respectively obtained by using the following three solutions of the prior art and the aspect of the present disclosure when the volunteer kept talking (that is, the mouth kept moving) during a brain imaging scan:

Solution one: Without performing motion estimation and correction, the existing SENSE method is used to perform image reconstruction to obtain a reconstructed image 101.

Solution two: The existing linear SAMER method is used to perform motion estimation, correction, and image reconstruction to obtain a reconstructed image 102.

Solution three: The solution provided in the aspect of the present disclosure is used to perform motion estimation, correction, and image reconstruction to obtain a reconstructed image 103.

It can be seen from 101-103 that the reconstructed image corresponding to 103 has clearer details.

FIG. 11 is a schematic diagram showing a comparison between rigid body motion vectors obtained by solution three and solution two in FIG. 10, where the abscissa is time, in units of s, and the ordinate is a parameter value of the rigid body motion vector, in units of mm (corresponding to a translation parameter) or mm/deg (corresponding to a rotation parameter). In the figure:

6 dashed lines of different colors in 1031 correspond to the rotation parameter values of the three directions and the translation parameter values of the three directions of rigid body motion vectors obtained by solution three, respectively. Specifically:
the dark blue dashed line corresponds to the rotation parameter value Rx in the x direction;
the red dashed line corresponds to the rotation parameter value Ry in the y direction;
the yellow dashed line corresponds to the rotation parameter value Rz in the z direction;
the purple dashed line corresponds to the translation parameter value Tx in the x direction;
the green dashed line corresponds to the translation parameter value Ty in the y direction;
the light blue dashed line corresponds to the translation parameter value Tz in the z direction.

6 solid lines of different colors in 1021 correspond to the rotation parameter values of the three directions and the translation parameter values of the three directions of rigid body motion vectors obtained by solution two, respectively. Specifically:
the dark blue solid line corresponds to the rotation parameter value Rx in the x direction;
the red solid line corresponds to the rotation parameter value Ry in the y direction;
the yellow solid line corresponds to the rotation parameter value Rz in the z direction;
the purple solid line corresponds to the translation parameter value Tx in the x direction;
the green solid line corresponds to the translation parameter value Ty in the y direction;
the light blue solid line corresponds to the translation parameter value Tz in the z direction.

In order to more clearly compare the rigid body motion vectors obtained by solution three and solution two in FIG. 11, FIG. 12 is a schematic diagram showing an enlargement of the rigid body motion vectors obtained by solution three and solution two for a time of 80-90 s in FIG. 11. It can be seen from FIGS. 10 and 12 that the rigid body motion vector obtained by solution three can correct the influence of the non-rigid body motion of the mouth on the overall rigid body motion of the head, reflecting the robustness of the aspect of the present disclosure.

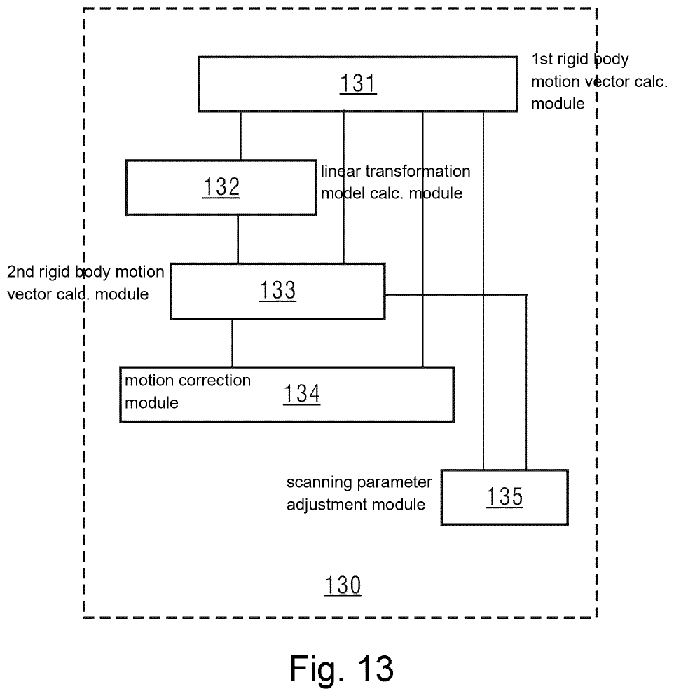

FIG. 13 is a schematic structural diagram of a motion estimation apparatus 130 for MR imaging provided in an aspect of the present disclosure. The apparatus 130 mainly includes: a first rigid body motion vector calculation module 131, a linear transformation model calculation module 132, a second rigid body motion vector calculation module 133, a motion correction module 134, and a scanning parameter adjustment module 135.

The first rigid body motion vector calculation module 131 is configured to calculate a rigid body motion vector of a subject for each of the most recent N imaging excitations, where N is a preset integer greater than 1.

The linear transformation model calculation module 132 is configured to: according to PT data of each channel acquired for the most recent N imaging excitations and N rigid body motion vectors calculated for the most recent N imaging excitations, calculate a linear transformation model for transforming from the PT data of each channel acquired for the most recent N imaging excitations to the N rigid body motion vectors; wherein a PT scan is continuously performed on the subject during an imaging scan of the subject.

The second rigid body motion vector calculation module 133 is configured to: calculate, for each subsequent imaging excitation, a rigid body motion vector of the subject for each echo of each imaging excitation according to PT data of each channel acquired during an echo duration of each echo and the linear transformation model.

In an optional aspect, the first rigid body motion vector calculation module 131 calculating the rigid body motion vector of the subject for each of the most recent N imaging excitations includes: acquiring scout data, wherein a scout scan is performed on the subject before the start of the first imaging scan of the subject; for each of the most recent N imaging excitations, acquiring PT data of each channel while acquiring imaging data, and also acquiring guidance data during an echo duration of a plurality of preset echoes of each imaging excitation; calculating the rigid body motion vector of the subject for each of the most recent N imaging excitations according to the scout data and the guidance data acquired for each of the most recent N imaging excitations.

In an optional aspect, the linear transformation model calculation module 132 calculating, according to the PT data of each channel acquired for the most recent N imaging excitations and the N rigid body motion vectors calculated for the most recent N imaging excitations, the linear transformation model for transforming from the PT data of each channel acquired for the most recent N imaging excitations to the N rigid body motion vectors includes: for each of the most recent N imaging excitations, selecting, from the PT data of each channel for the imaging excitation, PT data of each channel during a period from the start of acquiring the first piece of guidance data to the end of acquiring the last piece of guidance data, and averaging the PT data of each selected channel separately to obtain a PT mean value of each channel for the imaging excitation; according to the PT mean value of each channel for each of the most recent N imaging excitations and the N rigid body motion vectors, calculating the linear transformation model for transforming from the PT mean value of each channel for the imaging excitation to the N rigid body motion vectors.

In an optional aspect, the first rigid body motion vector calculation module 131 acquiring the PT data of each channel includes: acquiring PT data of each channel within a preset range of each echo center.

In an optional aspect, after acquiring the PT data of each channel, the first rigid body motion vector calculation module 131 further performs radio frequency interference suppression processing on the acquired PT data of each channel.

In an optional aspect, the second rigid body motion vector calculation module 133 calculating, for each subsequent imaging excitation, the rigid body motion vector of the subject for each echo of each imaging excitation according to the PT data of each channel acquired during the echo duration of each echo and the linear transformation model includes: for each echo, averaging the PT data of each channel corresponding to the echo to obtain the PT mean value of each channel for the echo, and calculating the rigid body motion vector of the subject for the echo according to the PT mean value of each channel for the echo and the linear transformation model.

In an optional aspect, the above-mentioned apparatus 130 further includes: a motion correction module 134, configured to: when all imaging excitations for the imaging scan of the subject have been completed, for any imaging excitation, when the rigid body motion vector of the subject for the imaging excitation is calculated by the first rigid body motion vector calculation module 131, perform motion correction on imaging data of the imaging excitation by using the rigid body motion vector for the imaging excitation; for any imaging excitation, when the rigid body motion vector of the subject for each echo of the imaging excitation is calculated by the second rigid body motion vector calculation module 133, perform motion correction on imaging data of each echo acquired for the imaging excitation by using the rigid body motion vector for the echo.

In an optional aspect, the above-mentioned apparatus 130 further includes: a scanning parameter adjustment module 135, configured to: after the first rigid body motion vector calculation module 131 calculates the rigid body motion vector of the subject for each of the most recent N imaging excitations, for each of the most recent N imaging excitations, according to the rigid body motion vector of the subject for the imaging excitation, or according to the rigid body motion vectors of the subject for the most recent M imaging excitations, predict a position to which the subject will move in a next imaging excitation; and adjust scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N; and/or, configured to: after the second rigid body motion vector calculation module 133 calculates the rigid body motion vector of the subject for each echo of each imaging excitation, for each imaging excitation, according to the rigid body motion vector of the subject for each echo of the imaging excitation, or according to the rigid body motion vector of the subject for each echo of the most recent Q imaging excitations, predict a position to which the subject will move in a next imaging excitation; and adjust scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

The aspects of the present disclosure further provide an MRI system, and the MRI system includes any one of the motion estimation apparatuses 130 for MR imaging described above.

In an optional aspect, the MR imaging system further includes: a PT transmitter placed at the back side of a subject's head.

It should be noted that the motion estimation method and apparatus for MR imaging, and the MR imaging system provided in the aspects of the present disclosure may be a method, apparatus, and system applied in medical imaging.

The aspects of the present disclosure further provide a computer program product, including a computer program or instructions, which, when executed by a processor, implements the steps of the motion estimation method for MR imaging as described in any one of the above aspects.

The aspects of the present disclosure further provide a computer-readable storage medium, wherein the computer-readable storage medium stores instructions, and the instructions, when executed by a processor, can perform the steps of the motion estimation method for MR imaging as described above. In practical applications, the computer-readable medium may be included in each device/apparatus/system in the above aspects or may exist independently without being assembled into the device/apparatus/system. Instructions are stored in the computer-readable storage medium, and the instructions stored therein, when executed by a processor, can perform the steps of the motion estimation method for MR imaging as described above.

The aspects of the present disclosure further provide an electronic device. The electronic device may include a processor with one or more processing cores, a memory with one or more computer-readable storage media, and a computer program stored in the memory and executable on the processor. When the program in the memory is executed, the motion estimation method for MR imaging described above may be implemented.

Those skilled in the art will understand that features stated in various aspects and/or claims of the present application can be combined and/or integrated in various ways, even if such combinations or integrations are not clearly stated in the present application. In particular, without departing from the spirit and teaching of the present application, features stated in aspects and/or claims of the present application can be combined and/or integrated in various ways, and all such combinations and/or integrations fall within the scope of disclosure of the present application.

Specific aspects have been used herein to expound the principles and forms of implementation of the present application, but the description of the aspects above is merely intended to help understand the method of the present application and the core idea thereof, not to restrict the present application. Those skilled in the art can make changes in terms of the specific form of implementation and the application scope, based on the idea, spirit, and principles of the present application, and any modifications, equivalent replacements, improvements, etc. made thereby should be included within the scope of protection of the present application.

1. A motion estimation method for magnetic resonance imaging, the method comprising:
calculating a rigid body motion vector of a subject for each of a most recent N imaging excitations, where N is a preset integer greater than 1;
calculating, according to pilot tone (PT) data of each channel acquired for the most recent N imaging excitations and N rigid body motion vectors calculated for the most recent N imaging excitations, a linear transformation model for transforming from the PT data of each channel acquired for the most recent N imaging excitations to the N rigid body motion vectors, wherein a PT scan is continuously performed on the subject during an imaging scan of the subject; and
for each subsequent imaging excitation, according to PT data of each channel acquired during an echo duration of each echo and the linear transformation model, calculating a rigid body motion vector of the subject for each echo of each imaging excitation.

2. The method as claimed in claim 1, wherein calculating the rigid body motion vector of the subject for each of the most recent N imaging excitations comprises:
acquiring scout data, wherein a scout scan is performed on the subject before a start of a first imaging scan of the subject;
for each of the most recent N imaging excitations, acquiring PT data of each channel while acquiring imaging data, and acquiring guidance data during an echo duration of a plurality of preset echoes of each imaging excitation; and
calculating the rigid body motion vector of the subject for each of the most recent N imaging excitations according to the scout data and the guidance data acquired for each of the most recent N imaging excitations.

3. The method as claimed in claim 2, wherein calculating the rigid body motion vector of the subject for each of the most recent N imaging excitations according to the scout data and the guidance data acquired for each of the most recent N imaging excitations comprises:
by using a linear scout accelerated retrospective motion estimation and reduction (SAMER) method, according to the scout data and the guidance data acquired for each of the most recent N imaging excitations, calculating the rigid body motion vector of the subject for each of the most recent N imaging excitations.

4. The method as claimed in claim 2, wherein calculating, according to the PT data of each channel acquired for the most recent N imaging excitations and N rigid body motion vectors calculated for the most recent N imaging excitations, the linear transformation model for transforming from the PT data of each channel acquired for the most recent N imaging excitations to the N rigid body motion vectors comprises:
for each of the most recent N imaging excitations, selecting, from the PT data of each channel for the imaging excitation, PT data of each channel during a period from the start of acquiring a first piece of guidance data to an end of acquiring the last piece of guidance data, and averaging the PT data of each selected channel to obtain a PT mean value of each channel for the imaging excitation; and
according to the PT mean value of each channel for each of the most recent N imaging excitations and the N rigid body motion vectors, calculating the linear transformation model for transforming from the PT data of each channel acquired for the most recent N imaging excitations to the N rigid body motion vectors.

5. The method as claimed in claim 1, wherein calculating, for each subsequent imaging excitation, the rigid body motion vector of the subject for each echo of each imaging excitation according to the PT data of each channel acquired during the echo duration of each echo and the linear transformation model comprises:
for each echo, averaging the PT data of each channel corresponding to the echo to obtain a PT mean value of each channel for the echo, and calculating the rigid body motion vector of the subject for the echo according to the PT mean value of each channel for the echo and the linear transformation model.

6. The method as claimed in claim 1, wherein after calculating the rigid body motion vector of the subject for each echo of each imaging excitation, the method further comprises, when all imaging excitations for the imaging scan of the subject have been completed:
for any imaging excitation, when the rigid body motion vector of the subject for the imaging excitation is calculated, performing motion correction on imaging data of the imaging excitation by using the rigid body motion vector for the imaging excitation; and
for any imaging excitation, when the rigid body motion vector of the subject for each echo of the imaging excitation is calculated, performing motion correction on imaging data of each echo acquired for the imaging excitation by using the rigid body motion vector for the echo.

7. The method as claimed in claim 1, wherein after calculating the rigid body motion vector of the subject for each of the most recent N imaging excitations, the method further comprises:
for each of the most recent N imaging excitations, according to the rigid body motion vector of the subject for the imaging excitation, or according to the rigid body motion vectors of the subject for the most recent M imaging excitations, predicting a position to which the subject will move in a next imaging excitation, and adjusting scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N; and/or
after calculating the rigid body motion vector of the subject for each echo of each imaging excitation, the method further comprises, for each imaging excitation, according to the rigid body motion vector of the subject for each echo of the imaging excitation, or according to the rigid body motion vector of the subject for each echo of the most recent Q imaging excitations, predicting a position to which the subject will move in a next imaging excitation, and adjusting scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

8. A motion estimation apparatus for magnetic resonance imaging, wherein the apparatus comprises:
a first rigid body motion vector calculation module, configured to calculate a rigid body motion vector of a subject for each of a most recent N imaging excitations, where N is a preset integer greater than 1;
a linear transformation model calculation module configured to calculate, according to pilot tone (PT) data of each channel acquired for the most recent N imaging excitations and N rigid body motion vectors calculated for the most recent N imaging excitations, a linear transformation model for transforming from the PT data of each channel acquired for the most recent N imaging excitations to the N rigid body motion vectors; wherein a PT scan is continuously performed on the subject during an imaging scan of the subject; and
a second rigid body motion vector calculation module, configured to calculate, for each subsequent imaging excitation, a rigid body motion vector of the subject for each echo of each imaging excitation according to PT data of each channel acquired during an echo duration of each echo and the linear transformation model.

9. The apparatus as claimed in claim 8, wherein the first rigid body motion vector calculation module calculating the rigid body motion vector of the subject for each of the most recent N imaging excitations comprises:
acquiring scout data, wherein a scout scan is performed on the subject before a start of the first imaging scan of the subject;
for each of the most recent N imaging excitations, acquiring PT data of each channel while acquiring imaging data, and also acquiring guidance data during an echo duration of a plurality of preset echoes of each imaging excitation; and
calculating the rigid body motion vector of the subject for each of the most recent N imaging excitations according to the scout data and the guidance data acquired for each of the most recent N imaging excitations.

10. The apparatus as claimed in claim 8, wherein the linear transformation model calculation module calculating, according to the PT data of each channel acquired for the most recent N imaging excitations and the N rigid body motion vectors calculated for the most recent N imaging excitations, the linear transformation model for transforming from the PT data of each channel acquired for the most recent N imaging excitations to the N rigid body motion vectors comprises:
for each of the most recent N imaging excitations, selecting, from the PT data of each channel for the imaging excitation, PT data of each channel during a period from a start of acquiring a first piece of guidance data to an end of acquiring the last piece of guidance data, and averaging the PT data of each selected channel to obtain a PT mean value of each channel for the imaging excitation; and
according to the PT mean value of each channel for each of the most recent N imaging excitations and the N rigid body motion vectors, calculating the linear transformation model for transforming from the PT mean value of each channel for the imaging excitation to the N rigid body motion vectors.

11. The apparatus as claimed in claim 8, wherein the second rigid body motion vector calculation module calculating, for each subsequent imaging excitation, the rigid body motion vector of the subject for each echo of each imaging excitation according to the PT data of each channel acquired during the echo duration of each echo and the linear transformation model comprises:
for each echo, averaging the PT data of each channel corresponding to the echo to obtain the PT mean value of each channel for the echo, and calculating the rigid body motion vector of the subject for the echo according to the PT mean value of each channel for the echo and the linear transformation model.

12. The apparatus as claimed in claim 8, wherein the apparatus further comprises: a motion correction module, configured to, when all imaging excitations for the imaging scan of the subject have been completed:
for any imaging excitation, when the rigid body motion vector of the subject for the imaging excitation is calculated, perform motion correction on imaging data of the imaging excitation by using the rigid body motion vector for the imaging excitation; and
for any imaging excitation, when the rigid body motion vector of the subject for each echo of the imaging excitation is calculated, perform motion correction on imaging data of each echo acquired for the imaging excitation by using the rigid body motion vector for the echo.

13. The apparatus as claimed in claim 8, wherein the apparatus further comprises a scanning parameter adjustment module, configured to:
after the first rigid body motion vector calculation module calculates the rigid body motion vector of the subject for each of the most recent N imaging excitations, for each of the most recent N imaging excitations, according to the rigid body motion vector of the subject for the imaging excitation, or according to the rigid body motion vectors of the subject for the most recent M imaging excitations, predict a position to which the subject will move in a next imaging excitation; and adjust scanning parameters of the next imaging excitation according to the position to which the subject will move in the next imaging excitation, so that a scanning area corresponding to the adjusted scanning parameters is matched with a target scanning area of the subject, where M is a preset integer greater than 1, and M≤N; and/or
after the second rigid body motion vector calculation module calculates the rigid body motion vector of the subject for each echo of each imaging excitation, for each imaging excitation, according to the rigid body motion vector of the subject for each echo of the imaging excitation, or according to the rigid body motion vector of the subject for each echo of the most recent Q imaging excitations, predict a position to which the subject will move in a next imaging excitation, and adjust scanning parameters of the next imaging excitation according to the position to which the subject will move in the next imaging excitation, so that a scanning area corresponding to the adjusted scanning parameters is matched with a target scanning area of the subject, where Q is a preset integer greater than 1.

14. A magnetic resonance imaging system, wherein the magnetic resonance imaging system comprises the motion estimation apparatus for magnetic resonance imaging as claimed in claim 8.
